(19) United States
(12) Patent Application Publication
(10) Pub. No.: US 2021/0353600 A1
(43) Pub. Date: Nov. 18, 2021

(54) COMPOSITIONS FOR TREATING HAIR LOSS

(71) Applicant: Lars BRICHTA, Brooklyn, NY (US)

(72) Inventor: Lars BRICHTA, Brooklyn, NY (US)

(73) Assignee: ChemistryRX, Folcroft, PA (US)

(21) Appl. No.: 17/302,777

(22) Filed: May 12, 2021

Related U.S. Application Data
(60) Provisional application No. 63/023,279, filed on May 12, 2020.

(51) Int. Cl. A61K 31/436; A61K 31/506; A61K 31/585; A61K 31/655; A61K 31/58; A61K 31/5575; A61K 31/573

(52) U.S. Cl. CPC A61K 31/436; A61K 31/506; A61K 31/585; A61K 31/573; A61K 31/58; A61K 31/5575; A61K 31/655

(57) ABSTRACT

Compositions and methods for treating hair loss in a patient by administering a composition having an active agent in a mixed solvent containing an aqueous phase and an oil phase are described herein.

### CROSS-REFEREBCE TO RELATED APPLICATIONS

This application claims priority from U.S. Provisional No. 63/023,279, entitled “Compositions for Treating Hair Loss,” filed May 12, 2020, the entirety of which is hereby incorporated by reference in its entirety.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Not applicable

### SUMMARY OF THE INVENTION

Various embodiments of the invention are directed to methods for treating hair loss comprising topically administering to a patient in need of treatment a liquid composition containing an active agent is selected from the group consisting of minoxidil, finasteride, dutasteride, tofacitinib, tacrolimus, bimatoprost, latanoprost, spironolactone, aldactone, kenalog-10, kenalog-40, triamcinolone, azulfidine, sulfasalazine, sulfazine, and the like and combinations thereof and a solvent. The patient in such methods may have a disorder such as alopecia areata, alopecia totalis, alopecia universalis, vitiligo, graft versus host disease, telogen effluvium, tinea capitis (dermatophytosis), hypotrichosis, hereditary hypotrichosis simplex, frontal fibrosing alopecia, cicatricial alopecia, lichen planopilaris, ring alopecia, and chemotherapy induced alopecia. In some embodiments, the active agent may be tacrolimus. In various embodiments, the active agent may have a concentration of about 0.25% (w/w) to about 15% (w/w) based on the total composition. In some embodiments, the solvent may be phosphate buffered saline solution, water, or saline, and in some embodiments, the solvent may be water, isopropyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), transcutol, propylene glycol (PG), N-methyl-2 pyrrolidone (NMP), methylene chloride, diethyl ether, ethanol, acetonitrile, ethyl acetate, benzyl alcohol, a combination of natural oil; ethylene glycol, propylene glycol, dimethyl polysiloxane (DMPX), oleic acid, caprylic acid, 1-octanol, ethanol (denatured or anhydrous), liposomal compositions, plant oils, Aloe vera derivatives or sesame seed oil or derivatives thereof, ethosomes, azone, castor oil derivatives, ethoxylated castor oil, jojoba oil derivatives, corn oil derivatives, emu oil derivatives, and and combinations thereof.

In certain embodiments, the solvent may be an oil in an aqueous solution. The oil in such embodiments may be coconut oil, squalane, isopropyl myristate, isopropyl isostearate, isopropyl palmitate, modified triglycerides, caprylic capric glycerides, fractionated triglycerides, glyceryl tricaprate, glyceryl tricaproate, glyceryl tricaprylate, glyceryl tricaprylate/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8-C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and mixtures thereof. In some embodiments, the oil may have a concentration of about 0.5% (w/w) to about 10% (w/w) based on the total weight of the composition. The aqueous phase may be water or a polar water-miscible solvent such as, for example, C1-C4 alcohols, polyethylene glycol, ethylene glycol, propylene glycol, butylene glycol, pentylene glycol, glycerol, diethylene glycol monoethyl ether, propylene carbonate, and combinations thereof. In some embodiments, the polar water-miscible solvent may have a concentration of about 0.5% (w/w) to about 10% (w/w) based on the total composition.

In some embodiments, the composition may further include about 0.1 wt % to about 5 wt % surfactant based on the total weight of the composition. The surfactant may be alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters, Span 20 or Span 80, block copolymers of ethylene oxides and propylene oxides, Pluronic L121 or Pluronic F68, polymeric surfactants having crosslinked copolymers of acrylic acid, Pemulen Tr-1 and Pemulen Tr-2, and combinations thereof in such embodiments. In some embodiments, the composition may further include about 1% (w/w) to about 20% (w/w) penetration enhancer based on the total weight of the composition. In some embodiments, the composition may include an antioxidant such as, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and pharmaceutically acceptable salt and ester thereof, and combinations thereof.

Further embodiments are directed to compositions containing an active agent such as minoxidil, finasteride, dutasteride, tofacitinib, tacrolimus, bimatoprost, latanoprost, spironolactone, aldactone, kenalog-10, kenalog-40, triamcinolone, azulfidine, sulfasalazine, sulfazine, and the like and combinations thereof and an oil in an aqueous solution.

The oil in such embodiments may be coconut oil, squalane, isopropyl myristate, isopropyl isostearate, isopropyl palmitate, modified triglycerides, caprylic capric glycerides, fractionated triglycerides, glyceryl tricaprate, glyceryl tricaprylate, glyceryl tricaprylate, glyceryl tricaprylate/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8-C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and mixtures thereof. In some embodiments, the oil may have a concentration of about 0.5% (w/w) to about 10% (w/w) based on the total weight of the composition. The aqueous phase may be water or a polar water-miscible solvent such as, for example, C1-C4 alcohols, polyethylene glycol, ethylene glycol, propylene glycol, butylene glycol, pentylene glycol, glycerol, diethylene glycol monoethyl ether, propylene carbonate, and combinations thereof. In some embodiments, the polar water-miscible solvent may have a concentration of about 0.5% (w/w) to about 10% (w/w) based on the total composition.

In some embodiments, the composition may further include about 0.1 wt % to about 5 wt % surfactant based on the total weight of the composition. The surfactant may be alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters, Span 20 or Span 80, block copolymers of ethylene oxides and propylene oxides, Pluronic L121 or Pluronic F68, polymeric surfactants having crosslinked copolymers of acrylic acid, Pemulen Tr-1 and Pemulen Tr-2, and combinations thereof in such embodiments. In some embodiments, the composition may further include about 1% (w/w) to about 20% (w/w) penetration enhancer based on the total weight of the composition. In some embodiments, the composition may include an antioxidant such as, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and pharmaceutically acceptable salt and ester thereof, and combinations thereof.

### DESCRIPTION OF THE DRAWINGS

Not applicable

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

All percentages, parts and ratios are based upon the total weight of the topical compositions and all measurements made are at about 25° C., unless otherwise specified.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 and 7 μm are also intended to be explicitly disclosed, as well as the range of values greater than or equal to 1 μm and the range of values less than or equal to 8 μm.

The singular forms “a,” “an,” and “the” include plural referents unless the context clearly dictates otherwise. Thus, for example,

Reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g. “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subj ect.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are--within the scope of sound medical judgment--suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “appreciable” is employed herein to refer to those agents of interest/compounds, salts, compositions, dosage forms, etc., which—within the scope of sound medical judgement—resulted in increased hair growth. An improvement in hair growth may be quantified by a SALT score or by a % regrowth measurement. A positive appreciable change in hair growth may not rise to a pharmaceutically acceptable or cosmetically acceptable determination.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

The term “SALT” refers to the Severity of Alopecia Tool, which is a statistical measurement that may be used by those skilled in the art to quantize change in the severity of alopecia in a patient or a sample of patients overall. A negative change in SALT score indicates an improvement in a subject's condition.

As used herein, the term “pharmaceutically acceptable carrier” refers to any of the standard pharmaceutical carriers including, but not limited to non-toxic solvent, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to liquid compositions containing one or more active agents such as, for example, minoxidil, finasteride, dutasteride, tofacitinib, tacrolimus, bimatoprost, latanoprost, spironolactone, aldactone, kenalog-10, kenalog-40, triamcinolone, azulfidine, sulfasalazine, sulfazine, and the like and combinations thereof. Such active agents can be provided in any amount capable of providing treatment. For example, the compositions of embodiments may include up to about 15% (w/w), about 0.25% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), or any range or individual concentration of active agent encompassed by these example ranges. Such liquid compositions may be administered to the scalp of a subject experiencing hair loss without the matting, weighing down, and oiliness associated with creams and ointments currently used to deliver active agents to the scalp.

Example compositions may include various known components. For example, in some embodiments, the composition may include a solvent such as water, isopropyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), transcutol, propylene glycol (PG), N-methyl-2 pyrrolidone (NMP), methylene chloride, diethyl ether, ethanol, acetonitrile, ethyl acetate, benzyl alcohol, a combination of natural oil; ethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, ethosomes, azone, castor oil derivatives, such as ethoxylated castor oil, jojoba oil derivatives, corn oil derivatives, emu oil derivatives, and the like and combinations thereof. The solvent can be present in a concentration of about 50% (w/w) to about 99.9% (w/w), about 60% (w/w) to about 99.8% (w/w), about 75% (w/w) to about 99.5% (w/w), about 80% (w/w) to about 100% (w/w), or any range or individual concentration of solvent encompassed by these example ranges.

In some embodiments, the solvent may be a mixture of solvents. For example, the composition may contain an oil in an aqueous solution. The one or more of the one or more active agents may be dissolved in the oil phase. Examples of oils that can be used in the compositions include coconut oil, squalane, isopropyl myristate, isopropyl isostearate, isopropyl palmitate, modified triglycerides, caprylic capric glycerides, fractionated triglycerides, glyceryl tricaprate, glyceryl tricaproate, glyceryl tricaprylate, glyceryl tricaprylate/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8-C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and the like and mixtures thereof. In various embodiments, the compositions may include an oil at a concentration of about 0.5% (w/w) to about 10% (w/w), about 1% (w/w) to about 5% (w/w), about 0.5% (w/w) to about 5% (w/w), or any range or individual concentration of solvent encompassed by these example ranges.

In some embodiments, the aqueous phase may be water. In other embodiments, the aqueous phase may include a polar water-miscible solvent, such as an alcohol or glycol. Polar water-miscible solvents may improve skin penetration and solvation of the active agent. The polar water-miscible solvent may be, for example, C₁-C₄ alcohols, polyethylene glycol, ethylene glycol, propylene glycol, butylene glycol, pentylene glycol, glycerol, diethylene glycol mono ethyl ether, propylene carbonate, and the like and combinations and mixtures thereof. The total amount of polar water-miscible solvent may be less than about 10 wt % by weight of the total composition, about 0.5% (w/w) to about 10% (w/w), about 1% (w/w) to about 5% (w/w), about 0.5% (w/w) to about 5% (w/w), or any range or individual concentration of solvent encompassed by these example ranges.

In some embodiments, the compositions may include a surfactant. The surfactant may be incorporated into the oil phases, the aqueous phase, or both. Suitable surfactants include, for example, alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters (such as Span 20 or Span 80), block copolymers of ethylene oxides and propylene oxides (such as Pluronic L121 or Pluronic F68), polymeric surfactants having crosslinked copolymers of acrylic acid, such as Pemulen Tr-1 and Pemulen Tr-2, and the like and combinations and mixtures thereof. The composition may include surfactant in a concentration of about 0.1 wt % to about 5 wt %, about 0.5 wt % to about 3 wt %, about 0.7 to about 2 wt %, or any range or individual concentration of solvent encompassed by these example ranges.

In some embodiments, the compositions may include an antioxidant. Such antioxidant may be, for example, butylated hydroxytoluene, ascorbic acid, ascorbic palmitate, butylated hydroxyani sole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. The amount of humectant in such compositions may be about 0.01% (w/w) to 5% (w/w) of the total composition.

In some embodiments, the composition may further include a UV-absorbing compound such as, for example, glyceryl PABA, padimate O, roxadimate, dioxybenzone, oxybenzone, sulisonbenzone, octocrylene, octyl methoxycinnamate, ethoxyethyl p-methoxycinnamate, homomenthyl salicylate, ethylhexyl salicylate, trolamine salicylate, avobenzone, ecamsule, ensulizole, bemotrizinol, bisoctrizole, and the like and combinations thereof. The amount of UV-absorbing compound may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

The compositions of the various embodiments described herein may include one or more penetration enhancers. The penetration enhancer in the compositions of various embodiments described above may be present in an amount about 0.5% (w/w) to about 40% (w/w), about 1% (w/w) to about 20% (w/w), about 5% (w/w) to about 15% (w/w) based on the total composition or any range or individual concentration encompassed by these example ranges.

In various embodiments, the aqueous phase may be about 20% (v/v) to about 90% (v/v), about 25% (v/v) to about 80% (v/v), about 30% (v/v) to about 75% (v/v) based on the total volume of the composition, or any range or individual concentration encompassed by these example ranges. The remainder of the composition may be the oil phase or a combination of oil phases.

Other embodiments of the invention include methods for treating hair growth disorders or hair loss by administering the compositions described above. Such methods are not limited to particular indications; however, the compositions described herein can be particularly useful for treating alopecia areata, alopecia totalis, alopecia universalis, vitiligo, and graft versus host disease. Other indications that can be treated by administering the compositions of various embodiments, include telogen effluvium, tinea capitis (dermatophytosis), hypotrichosis, hereditary hypotrichosis simplex, frontal fibrosing alopecia, cicatricial alopecia, lichen planopilaris, ring alopecia, chemotherapy induced alopecia, and the like.

The methods of various embodiments may include the steps of administering compositions described above to an area of skin on a subject in need of treatment. In some embodiments, the step of administering can be carried out one or two times per week, one, two, or three times per month, one to four times per year, and the like and any time period encompassed by these examples. In certain embodiments, administering can be carried out the prescribed number of times per day for one week to six months.

The step of administering can be carried out by various means. For example, administering can be accomplished by injecting the composition to the subdermal and subcutaneous areas of the scalp in need of treatment. In some embodiments, administering may include applying mechanical force or energy to the skin of the subject to facilitate delivery. For example, administering includes injecting the composition into the skin of the subject using microneedles. In further embodiments, administering can be carried out using a tattoo machine or other machines for subcutaneously injecting substances into a subject. As is known in the art, certain means for administering may require the use of particular components of the formulation. Such components are described above and can be appropriately incorporated into the compositions.

1. A method for treating hair loss comprising topically administering to a patient in need of treatment a liquid composition containing an active agent is selected from the group consisting of minoxidil, finasteride, dutasteride, tofacitinib, tacrolimus, bimatoprost, latanoprost, spironolactone, aldactone, kenalog-10, kenalog-40, triamcinolone, azulfidine, sulfasalazine, sulfazine, and the like and combinations thereof and a solvent.

2. The method of claim 1, wherein the active agent is tacrolimus.

3. The method of claim 1, wherein the active agent has a concentration of about 0.25% (w/w) to about 15% (w/w).

4. The method of claim 1, wherein solvent is phosphate buffered saline solution, water, or saline.

5. The method of claim 4, wherein the active agent, and phosphate buffered saline solution, water, or saline are the only components of the formulation.

6. The method of claim 1, wherein the solvent is selected from the group consisting of water, isopropyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), transcutol, propylene glycol (PG), N-methyl-2 pyrrolidone (NMP), methylene chloride, diethyl ether, ethanol, acetonitrile, ethyl acetate, benzyl alcohol, a combination of natural oil; ethylene glycol, propylene glycol, dimethyl polysiloxane (DMPX), oleic acid, caprylic acid, 1-octanol, ethanol (denatured or anhydrous), liposomal compositions, plant oils, Aloe vera derivatives or sesame seed oil or derivatives thereof, ethosomes, azone, castor oil derivatives, ethoxylated castor oil, jojoba oil derivatives, corn oil derivatives, emu oil derivatives, and and combinations thereof.

7. The method of claim 1, wherein the solvent is an oil in an aqueous solution.

8. The method of claim 8, wherein the oil is selected from the group consisting of coconut oil, squalane, isopropyl myristate, isopropyl isostearate, isopropyl palmitate, modified triglycerides, caprylic capric glycerides, fractionated triglycerides, glyceryl tricaprate, glyceryl tricaproate, glyceryl tricaprylate, glyceryl tricaprylate/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8-C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and mixtures thereof.

9. The method of claim 8, wherein the oil has a concentration of about 0.5% (w/w) to about 10% (w/w) based on the total compositions

10. The method of claim 8, wherein the aqueous phase is water or a polar water-miscible solvent.

11. The method of claim 12, wherein the aqueous phase is selected from the group consisting of C1-C4 alcohols, polyethylene glycol, ethylene glycol, propylene glycol, butylene glycol, pentylene glycol, glycerol, diethylene glycol monoethyl ether, propylene carbonate, and combinations thereof.

12. The method of claim 12, wherein polar water-miscible solvent has concentration of about 0.5% (w/w) to about 10% (w/w) based on the total composition.

13. The method of claim 1, wherein the composition further comprises about 0.1 wt % to about 5 wt % surfactant based on the total composition.

14. The method of claim 15, wherein the surfactant is selected from the group consisting of alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters, Span 20 or Span 80, block copolymers of ethylene oxides and propylene oxides, Pluronic L121 or Pluronic F68, polymeric surfactants having crosslinked copolymers of acrylic acid, Pemulen Tr-1 and Pemulen Tr-2, and combinations thereof.

15. The method of claim 1, further comprising about 1% (w/w) to about 20% (w/w) penetration enhancer based on the total composition.

16. The method of claim 1, further comprising an antioxidant selected from the group consisting of butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and pharmaceutically acceptable salt and ester thereof, and combinations thereof.

17. The method of claim 1, wherein the patient has a disorder selected from the group consisting of alopecia areata, alopecia totalis, alopecia universalis, vitiligo, graft versus host disease, telogen effluvium, tinea capitis (dermatophytosis), hypotrichosis, hereditary hypotrichosis simplex, frontal fibrosing alopecia, cicatricial alopecia, lichen planopilaris, ring alopecia, and chemotherapy induced alopecia.

18. A composition comprising an active agent is selected from the group consisting of minoxidil, finasteride, dutasteride, tofacitinib, tacrolimus, bimatoprost, latanoprost, spironolactone, aldactone, kenalog-10, kenalog-40, triamcinolone, azulfidine, sulfasalazine, sulfazine, and the like and combinations thereof and an oil in an aqueous solution.

19. The composition of claim 18, wherein the oil is selected from the group consisting of coconut oil, squalane, isopropyl myristate, isopropyl isostearate, isopropyl palmitate, modified triglycerides, caprylic capric glycerides, fractionated triglycerides, glyceryl tricaprate, glyceryl tricaproate, glyceryl tricaprylate, glyceryl tricaprylate/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8-C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and mixtures thereof.

20. The composition of claim 18, wherein the aqueous phase is water or a polar water-miscible solvent selected from the group consisting of water, C1-C4 alcohols, polyethylene glycol, ethylene glycol, propylene glycol, butylene glycol, pentylene glycol, glycerol, diethylene glycol monoethyl ether, propylene carbonate, and combinations thereof.
